SEQUENCE LISTING (7 sequences)

SEQ ID NO: 1 - protein, 26 aa, Unknown
  Synthetic peptide
GSGTMENLSRRLKVTGDLFDIMSGQT

SEQ ID NO: 2 - protein, 26 aa, Unknown
  synthetic peptide
GSGTMENLSRRLKVTEALFDIMSGQT

SEQ ID NO: 3 - protein, 200 aa, Homo sapiens
MSQSNRELVVDFLSYKLSQKGYSWSQFSDVEENRTEAPEGTESEMETPSAINGNPSWHLADSPAVNGATGHSSSLDAREVIPMAAVKQALREAGDEFELRYRRAFSDLTSQLHITPGTAYQSFEQVVNELFRDGVNWGRIVAFFSFGGALCVESVDKEMQVLVSRIAAWMATYLNDHLEPWIQENGGWDTFVELYGNAAA

SEQ ID NO: 4 - protein, 207 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMR

SEQ ID NO: 5 - protein, 146 aa, Kaposi′s sarcoma-associated herpesvirus
MDEDVLPGEVLAIEGIFMACGLNEPEYLYHPLLSPIKLYITGLMRDKESLFEAMLANVRFHSTTGINQLGLSMLQVSGDGNMNWGRALAILTFGSFVAQKLSNEPHLRDFALAVLPVYAYEAIGPQWFRARGGWRGLKAYCTQVLT

SEQ ID NO: 6 - protein, 166 aa, Human herpesvirus 4
MAYSTREILLALCIRDSRVHGNGTLHPVLELAARETPLRLSPEDTVVLRYHVLLEEIIERNSETFTETWNRFITHTEHVDLDFNSVFLEIFHRGDPSLGRALAWMAWCMHACRTLCCNQSTPYYVVDLSVRGMLEASEGLDGWIHQQGGWSTLIEDNIPGDDDDLE

SEQ ID NO: 7 - protein, 131 aa, murine herpesvirus 68
SGTYWATLITAFLKTVSKVEELDCVDSAVLVDVSKIITLTQEFRRHYDSVYRADYGPALKNWKRDLSKLFTSLFVDVINSGRIVGFFDVGRYVCEEVLCPGSWTEDHELLNDCMTHFFIENNLMNHFPLED

(19) United States
(12) Patent Application Publication
(10) Pub. No.: US 2016/0272691 A1
(43) Pub. Date: Sep. 22, 2016

(54) COMPOSITIONS AND METHODS FOR TREATING CONDITIONS ASSOCIATED WITH y-HERPESVIRUSES

(71) Applicant: NDSU RESEARCH FOUNDATION, Fargo, ND (US)

(72) Inventor: Sangita SINHA, Fargo, ND (US)

(21) Appl. No.: 15/021,996

(22) PCT Filed: Sep. 18, 2014
(86) PCT No.: PCT/US14/56324

§ 371 Date: Mar. 15, 2016

Related U.S. Application Data
(60) Provisional application No. 61/881,642, filed on Sep. 24, 2013.
(60) Provisional application No. 61/879,289, filed on Sep. 18, 2013.

(51) Int. Cl. C07K 14/47

(52) U.S. Cl. CPC C07K 14/4747; A61K 38/00

(57) ABSTRACT

One object of certain embodiments of the present invention is therapeutic agents and compositions to treat conditions associated with activation of yHV. In certain embodiments, the therapeutic agent is a synthetic peptide that preferentially binds to a viral Bcl-2 homolog over a cellular Bcl-2 homolog. In certain embodiments, the therapeutic agent is provided within a therapeutic composition including a pharmaceutically acceptable carrier or excipient for administering the synthetic peptide. The synthetic peptide may be administered by any suitable mode of administration, including, but not limited to, enteral or parenteral administration. In certain embodiments, the synthetic peptide may be comprised within a delivery vehicle.

### CROSS REFERENCE TO RELATED PATENTS

This application claims priority to U.S. Provisional Application No. 61/879,289, filed Sep. 18, 2013, and to U.S. Provisional Application No. 61/881,642, filed Sep. 24, 2013, each of which is incorporated by reference in its entirety.

### GOVERNMENT INTEREST STATEMENT

This invention was made with government support under NIH grants R21A0178108 and P20-RR015566, and NSF grants HRD-0811239 and EPS-0814442. The United States government has certain rights in the invention.

### FIELD OF THE INVENTION

The present invention relates generally to compositions and methods for treating conditions associated with activation of γHV. The therapeutic compositions include synthetic peptides that preferentially bind to viral Bcl-2 homologs over cellular Bcl-2 homologs.

### BACKGROUND OF THE INVENTION

γ-Herpesviruses (γHVs) are important human pathogens that infect 95% of all adults. Epstein-Barr virus (EBV), first isolated from Burkitt's lymphoma, is the causative agent for infectious mononucleosis and has been detected in several malignant tumors originating in both lymphoid and epithelial tissues. Kaposi's sarcoma-associated herpesvirus (KSHV), which is directly involved in the etiology of Kaposi sarcoma tumors, shows a high incidence among immunocompromised individuals, including people with HIV infection and transplant recipients. Another mammalian γHV, murine γHV68, does not infect humans, but provides the best model for studying human γHV infections in vivo. All γHVs encode mimics of the anti-apoptotic, cellular Bcl-2 proteins (cBcl-2s), which indicates that these proteins play an important role in the pathogenesis of these viruses.

Bcl-2 was the first cellular protein shown to function as an oncogene by blocking apoptotic cell death rather than by increasing cellular proliferation. Bcl-2 family members have now been shown to be multifunctional proteins influencing cellular processes ranging from autophagy, cell cycle progression, calcineurin signaling, and glucose homeostasis to transcriptional regulation. Members of the Bcl-2 family are identified by the presence of different Bcl-2 homology (BH) domains. This family includes several pro-apoptotic, BH3-only proteins like BIM and BAD, pro-apoptotic, multi-domain (BH3, BH1 and BH2) proteins like BAX and BAK, and multi-BH domain (BH4, BH3, BH1 and BH2) anti-apoptotic proteins like Bcl-2 and Bcl-X_{L}.

The γHV Bcl-2s have been shown to be critical for viral reactivation from latency and replication in immunocompromised hosts. Thus, they play important roles in latent and chronic infection. The γHV Bcl-2s also increase the probability of oncogenic transformation of infected cells by interfering with established tumor suppressor pathways and blocking premature cell death. The γHV Bcl-2s have anti-apoptotic and anti-autophagic functions. KSHV Bcl-2 blocks apoptosis stimulated by Bax or v-Cyclin overexpression or Sindbis virus infection, however, in cellular assays it does not appear to heterodimerize with pro-apoptotic Bcl-2 family members such as Bax and Bak. Further, KSHV Bcl-2 inhibits autophagy by binding to an essential autophagy effector, Beclin 1. EBV encodes two Bcl-2 homologs, BHRF1 and BALF1, each of which is expressed as early lytic cycle proteins. BHRF1 has anti-apoptotic activity and also disrupts the differentiation of epithelial cells, but the function of BALF1 remains unclear. Lastly, the γHV68 Bcl-2 homolog, M11, has been shown to inhibit apoptosis induced by Fas, TNFα, and Sindbis virus infection. Like KSHV Bcl-2, M11 also binds to Beclin 1 and inhibits autophagy. M11 is the only γHV Bcl-2 that has been demonstrated to play a role during infection in vivo. Thus, γHV and cBcl-2s are dual regulators of autophagy and apoptosis, and serve as a node of cross-talk between these pathways.

All anti-apoptotic Bcl-2s have similar three-dimensional structures that include a central hydrophobic α-helix surrounded by six or seven amphipathic helices. Structural and mutagenic analyses has demonstrated that the amphipathic, helical BH3 domain (BH3D) of pro-apoptotic proteins, as well as the pro-autophagic effector Beclin 1, binds to a hydrophobic surface groove on anti-apoptotic cBcl-2s and γHV Bcl-2s. Although human Bcl-2 paralogs share less than 50% sequence identity, known human and mice cellular Bcl-2 ortholog pairs share >85% sequence identity, with almost all residues lining the BH3D-binding groove being very highly conserved between orthologs. In contrast, despite their structural and functional similarity, the γHV Bcl-2s share very low sequence identity with each other and with cBcl-2s (FIG. 6). In fact, only the BH1 domain is well conserved in γHV Bcl-2s. For each Bcl-2, differences in amino acid sequence result in varying specificity for BH3 domains from different pro-apoptotic proteins.

There is a need in the art for compositions that selectively inhibit down-regulation of autophagy by viral Bcl-2 homologs, and methods for selectively inhibiting down-regulation of autophagy by viral Bcl-2 homologs.

### SUMMARY OF THE INVENTION

One object of certain embodiments of the present invention is therapeutic agents and corripositions to treat conditions associated with activation of γHV.

In certain embodiments, the therapeutic agent is a synthetic peptide that preferentially binds to a viral Bcl-2 homolog over a cellular Bcl-2 homolog. In certain embodiments, the therapeutic agent is provided within a therapeutic composition including a pharmaceutically acceptable carrier or excipient for administering the synthetic peptide. The synthetic peptide may be administered by any suitable mode of administration, including, but not limited to, enteral or parenteral administration. In certain embodiments, the synthetic peptide may be comprised within a delivery vehicle, including, for example, micelles, liposomes, and nanoparticles.

In certain embodiments, the affinity of the synthetic peptide for a viral Bcl-2 homolog is at least five times higher than its affinity for a cellular Bcl-2 homolog. In certain embodiments, the affinity of the synthetic peptide for a viral Bcl-2 homolog is at least ten times higher than its affinity for a cellular Bcl-2 homolog. In certain embodiments, the affinity of the synthetic peptide for a viral Bcl-2 homolog is at least 25 times higher than its affinity for a cellular Bcl-2 homolog. In certain embodiments, the affinity of the synthetic peptide for a viral Bcl-2 homolog is at least 50 times higher than its affinity for a cellular Bcl-2 homolog. In certain embodiments, the affinity of the synthetic peptide for a viral Bcl-2 homolog is at least 100 times higher than its affinity for a cellular Bcl-2 homolog. In certain embodiments, the dissociation constant (K_{d}) of the synthetic peptide for a viral Bcl-2 homolog is at least five, ten, 25, 50 or 100 times lower than its K_{d} for a cellular Bcl-2 homolog. In certain embodiments, the synthetic peptide has a K_{d} of 50 μM or less for a viral Bcl-2 homolog. In certain embodiments, the synthetic peptide does not detectably bind to a cellular Bcl-2.

In certain embodiments, the synthetic peptide alters the effect of viral Bcl-2 on a cellular pathway. In certain embodiments, the cellular pathway is autophagy or apoptosis. In certain embodiments, the synthetic peptide inhibits down-regulation of autophagy and/or apoptosis by a viral Bcl-2 homolog.

In certain embodiments the synthetic peptide is a mutant of a BH3 domain comprising substitutions at one or more amino acid positions. In certain embodiments, the synthetic peptide comprises a substitution at one or more amino acid positions conserved among BH3 domains. In certain embodiments, the synthetic peptide comprises a substitution at one or more amino acid positions conserved among BH3 domains that changes a hydrophobic amino acid or a basic amino acid to an alanine residue. In certain embodiments, the synthetic peptide comprises a substitution at one or more amino acid positions conserved among BH3 domains that changes a glycine residue to a polar or acidic amino acid.

In certain embodiments, the synthetic peptide comprises one or more amino acid substitutions relative to wild type (WT) Beclin 1 BH3 domain. In certain embodiments, the synthetic peptide comprises one or more amino acid substitutions relative to WT Beclin 1 BH3 domain at amino acid position L112, L116, K117, G120, D121, and F123. In certain embodiments, the synthetic peptide comprises one or more amino acid substitutions relative to WT Beclin 1 BH3 selected from L112A, L116A, K117A, G120E, D121A, and F123A.

In certain embodiments, the synthetic peptide homolog of Beclin 1 BH3 domain comprises one or more substitutions corresponding to position L8, L12, K13, G16, D17, or F19 of SEQ ID NO:1.

In certain embodiments, the synthetic peptide homolog of Beclin 1 BH3 domain comprises substitutions G16E and D17A relative to SEQ ID NO:1.

In certain embodiments, the synthetic peptide comprises SEQ ID NO:2 or its reverse sequence.

In certain embodiments, the synthetic peptide comprises at least one D amino acid.

In certain embodiments, one or more peptide bonds of the synthetic peptide is substituted with a non-peptide bond.

In certain embodiments, the synthetic peptide further comprises a cell-penetrating peptide sequence.

In certain embodiments, the synthetic peptide further comprises a cell-targeting moiety to target specific cell types. In certain embodiments, the cell-targeting moiety is an antibody to a cell surface antigen or a ligand for a receptor.

Another object of certain embodiments of the present invention is to provide methods of treating a person infected with a γHV in need of treatment, including a person infected with EBV or KSHV, by administering to the person an amount of the therapeutic agent of the invention effective to ameliorate one or more symptoms of the γHV infection. In certain embodiments is provided a method of treating infectious mononucleosis, chronic EBV disease, Burkitt's lymphoma, Hodgkins' lymphoma, nasopharyngeal carcinomas, gastric carcinoma, lymphomatoid granulomatosis, and numerous other lymphomas. In certain embodiments is provided a method of treating primary effusion lymphomas and Kaposi's sarcomas.

In certain embodiments, the method includes administering to a person in need of treatment of a condition associated with down-regulation of autophagy by viral BCL-2 homologs an amount of therapeutic agent effective to inhibit down-regulation of autophagy by viral BCL-2 homologs.

In certain embodiments, the therapeutic agent used in the method of the invention includes a synthetic peptide that is mutant of a BH3 domain comprising substitutions at one or more amino acid positions. In certain embodiments, the method uses a synthetic peptide comprising a substitution at one or more amino acid positions conserved among BH3 domains. In certain embodiments, the synthetic peptide used in the method of the invention comprises a substitution at one or more amino acid positions conserved among BH3 domains that changes a hydrophobic amino acid or a basic amino acid to an alanine residue. In certain embodiments, the synthetic peptide comprises a substitution at one or more amino acid positions conserved among BH3 domains that changes a glycine residue to a polar or acidic amino acid.

In certain embodiments, the methods employ a synthetic peptide comprising one or more amino acid substitutions relative to wild type (WT) Beclin 1 BH3 domain. In certain embodiments, the synthetic peptide comprises one or more amino acid substitutions relative to WT Beclin 1 BH3 domain at amino acid position L112, L116, K117, G120, D121, and F123. In certain embodiments, the synthetic peptide used in the method of the invention comprises one or more amino acid substitutions relative to WT Beclin 1 BH3 selected from L112A, L116A, K117A, G120E, D121A, and F123A.

In certain embodiments, the therapeutic agent used in the method of the invention is a synthetic peptide homolog of Beclin 1 BH3 domain comprising one or more substitutions corresponding to position L8, L12, K13, G16, D17, or F19 of SEQ ID NO:1. In certain embodiments, the synthetic peptide homolog of Beclin 1 BH3 domain comprises substitutions G16E and D17A relative to SEQ ID NO:1.

In certain embodiments, the therapeutic agent used in the method of the invention comprises SEQ ID NO:2 or its reverse sequence.

In certain embodiments, the method employs a synthetic peptide comprising at least one D amino acid.

In certain embodiments, the method employs a synthetic peptide having one or more peptide bonds substituted with a non-peptide bond.

In certain embodiments, the method employs a synthetic peptide further comprising a cell-penetrating peptide sequence.

In certain embodiments, the method employs a synthetic peptide further comprising a cell-targeting moiety.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

The preferred embodiments of the invention will be described in conjunction with the appended drawings provided to illustrate and not to the limit the invention.

FIGS. 1A and 1B are plots of the number of discrete GFP-LC3 punctae per cell in GFP-positive MCF7 cells co-transfected with GFP-LC3, WT M11 (FIG. 1A) or Bcl-X_{L}. (FIG. 1B) and WT or mutant Beclin 1.

FIG. 2 is an amino acid sequence alignment between Hs BclX1, Hs Bcl2, KSHV Bcl2, EBV BHRF1, and GHV68_H11 sequences.

### DETAILED DESCRIPTION OF EMBODIMENTS OF THE INVENTION

As described herein, the interaction of Beclin 1 with M11 and Bcl-X_{L} was used as a model system to systematically investigate and compare the roles of various interacting residues in binding of Beclin 1 to these two Bcl-2 homologs. First, autophagy levels in cells expressing different Beclin 1 mutants were quantified to identify Beclin 1 BH3D residues critical for down-regulation of autophagy by Bcl-X_{L}, but not by M11. Based on this information, isothermal titration calorimetry was used to identify a Beclin 1 BH3D-derived peptide that binds selectively to M11, but not to Bcl-X_{L}. Further, the crystal structure of this peptide bound to M11 was determined in order to elucidate the mechanism by which it binds selectively to M11. A comparison with the structure of the Beclin 1 BH3D bound to M11 shows that compensatory shifts of the peptide residue positions enable alternate interactions with M11. Lastly, it was demonstrated that in cells expressing normal Beclin 1, treatment with a cell-permeable version of the Beclin 1 BH3D-derived peptide results in abrogation of M11-mediated down-regulation of autophagy, but does not affect Bcl-X_{L}-mediated down-regulation of autophagy. These combined results help explain the atomic bases of the differential specificity of M11 and Bcl-X_{L}, and provide direct information on how M11-BH3D interactions may be targeted in vivo. This information is key to the rational design of inhibitors that selectively target M11, which would be invaluable in studying the interactions and roles of γHV Bcl-2s in cell culture and in vivo. Thus, these results will substantially inform future research on the γHVs, and may lead to novel therapeutics to treat γHV infections.

In certain embodiments, the invention provides compositions that selectively inhibit down-regulation of autophagy by viral Bcl-2 homologs. In certain embodiments, the composition is or comprises a synthetic homolog of the Beclin1 BH3 domain, which has the amino acid sequence GSGTMENLSRRLKVTGDLFDIMSGQT (SEQ ID NO:1). In certain embodiments, the synthetic homolog of Beclin 1 BH3 domain comprises substitutions at amino acids corresponding to positions 16 and 17 of SEQ ID NO:1, which in turn correspond to amino acid positions 120 and 121 of full length Beclin1. In certain embodiments, at least one of the substitutions at corresponding to positions 16 and 17 of SEQ ID NO:1 is a polar substitution. In certain embodiments, at least one polar substitution corresponding to positions 16 and 17 includes G16E, G16Q, or G16N of SEQ ID NO:1. In certain embodiments, the synthetic homolog of the Beclin1 BH3 domain comprises an amino acid substitution corresponding to E17A. In certain embodiments, the synthetic homolog of the Beclin1 BH3 domain comprises amino acid substitutions G16E, G16Q, or G16N and E17A. In certain embodiments, the synthetic homolog of the Beclin1 BH3 domain comprises the amino acid sequence GSGTMENLSRRLKVTEALFDIMSGQT (SEQ ID NO:2). In certain embodiments, the synthetic homolog of the Beclin1 BH3 domain comprises a substitution at a position corresponding to amino acid position L8, L12, and/or F19 of SEQ ID NO:1 or SEQ ID NO:2, which correspond to amino acid positions L112, L116, and F123 of full length Beclin1, respectively. In certain embodiments, the synthetic homolog of the Beclin1 BH3 domain comprises at least 7 amino acids. In certain embodiments, the synthetic homolog of Beclin1 BI-13 domain comprises at least at least 7 amino acids corresponding to 7 contiguous amino acids of SEQ ID NO:1, including substitutions at amino acids corresponding to amino acids G16 and D17 of SEQ ID NO:1. In certain embodiments, the synthetic homolog of Beclin1 BH3 domain includes a cell penetrating peptide. In certain embodiments, the cell penetrating peptide is a TAT peptide. In certain embodiments, the synthetic homolog of Beclin1 BH3 domain is a peptidomimetic comprising one or more D-amino acids and/or one or more non-hydrolyzable bonds (i.e., amino acid substituents that are covalently linked by a non-peptide bond). In certain embodiments, the synthetic homolog of Beclin1 BH3 domain has an amino acid sequence corresponding to the reverse sequence of SEQ. ID NO:1.

Certain embodiments provide methods for selectively inhibiting down-regulation of autophagy by viral Bcl-2 homologs by delivering to an organism infected with a virus an inhibitor that selectively inhibits down-regulation of autophagy by viral Bcl-2 homologs relative to inhibition of down-regulation of autophagy by cellular Bcl-2. In certain embodiments, the inhibitor used to inhibit down-regulation of autophagy by viral Bcl-2 homologs relative to inhibition of down-regulation of autophagy by cellular Bcl-2 is a synthetic homolog of the Beclin1 BH3 domain.

### Experimental Procedures

Protein Expression and Purification.

Various Bcl-2 homologs lacking the C-terminal transmembrane helix were cloned and expressed to enable purification of soluble constructs identical to those used for previous structural studies. γHV68 M11 residues 1-136 was expressed and purified. The double mutant variant of Bcl-X_{L} (N52D, N66D) was created by two rounds of site-directed mutagenesis using the QuikChange II Site-Directed Mutagenesis Kit (Agilent Technologies), then cloned, along with a C-terminal His6-tag for purification, into the NdeI and NotI restriction sites of pET 29b. The His6-tagged Bcl-X_{L} (residues 1-208, N52D, N66D) were expressed in E. coli strain BL21(DE3)pLysS, and soluble protein in the cell lysate was purified to homogeneity by Immobilized Metal Affinity Chromatography using two 5 mL His-Select columns (GE Healthcare) linked in tandem, followed by Ion-Exchange Chromatography using a Mono Q HR 10/10 column (GE Healthcare) and Size-Exclusion Chromatography using a 16/60 preparative Superdex 75 column (GE Healthcare).

Peptide Synthesis.

Various Beclin 1 BH3D-derived peptides, were chemically synthesized, and HPLC purified to >95% purity, with peptide purity confirmed by electrospray mass spectrometry (EZBioLabs/RSSynthesis/Protein Chemistry Technology Core at the University of Texas Southwestern Medical Center, Dallas).

Isothermal Titration Calorimetry:

Isothermal titration calorimetry was performed using a Nano ITC Low Volume (TA Instruments). For all ITC experiments, samples were loaded into separate dialysis cassettes, and dialyzed into ITC buffer. The ITC buffer for all experiments involving γHV68 M11 was 50 mM HEPES, pH 7.5, 250 mM NaCl and 2 mM β-mercaptoethanol and for human Bcl-X_{L} was 25 mM HEPES, pH 7.5, 100 mM NaCl and 2 mM β-mercaptoethanol. ITC was performed at 25° C. with 25 injections of 2 μL each. Data were analyzed using NanoAnalyze Software (TA Instruments), with an independent model.

Crystallization.

The M11+DM peptide complex was crystallized at 20° C. by hanging-drop vapor diffusion from a 1:1 mixture of protein stock and well solution (2.5 M (NH₄)₂SO₄ and 8% 2-propanol). Plate-shaped crystals were harvested and cryoprotected in a cryosolution consisting of 2.5 M (NH₄)₂SO₄ and 25% glycerol, and then immediately flash-frozen in liquid N₂.

Data Collection, Structure Solution and Refinement.

Diffraction intensities from one such crystal were recorded at 100 K from 1 sec. exposures over 0.5° crystal rotation per image, on a 4×4 tiled MARmosaic CCD detector (Rayonix), at a crystal to detector distance of 250 mm at beamline 23ID-D of GMCA@APS, ANL, Chicago. The data used to solve the structure were collected at 0.97934 Å in a sweep of 360° from a single crystal. Data were processed using HKL3000. Data statistics are summarized in Table 2.

Crystals belonged to the space group C2₁, with unit cell parameters of a=70.6 Å, b=140.8 Å, c=54.0 Å and β=127.8°, containing two copies of the M11-DM peptide complex per asymmetric unit. The positions and orientations of the two M11 (1-136) molecules, monomer A and B, were determined by molecular replacement using HKL3000/MOLREP, using a search model comprising a single M11 monomer lacking residues 52-73 of PDB ID: 3DVU. A helix corresponding to Beclin 1 BH3D (from PDB ID: 3DVU, chain C), with D124 mutated to Ala, was manually placed into appropriate density next to monomer A using the program Coot. A Glu side chain was built into appropriate electron density at position 120 after the first cycle of refinement. The NCS operator required to superimpose M11 monomer A onto B, was used to place a second copy of the Beclin 1 helix, peptide D, into appropriate density next to monomer B. The model was refined in the program refmac using imperfect two-fold NCS restraints (Table 3). The final model is deposited in the PDB with accession code 4M18.

Autophagy Assay.

Quantification of the fluorescent autophagosomes in MCF7 cells co-transfected with GFP-LC3 (1.6 μg), Beclin 1 (1.2 μg), and either Bcl-X_{L} (1.2 μg) or M11(1.2 μg) expression plasmids (4 μg plasmids total) was done using an inverted AxioObserver (Zeiss). Cells were cultured in DMEM with 10% fetal calf serum (growth medium) in 8-well slides (Millipore) and transfected at 80% confluence with Lipofectamine (Invitrogen). After transfection, cells were either starved overnight in Earle's balanced salt solution (EBSS, starvation medium), or grown in nutrient-rich media with the addition of 2×EAA (essential amino acids) and 2×NEAA (nonessential amino acids) (nutrient rich medium) in the absence or presence of 1 mM peptides if indicated. The number of GFP-LC3 puncta per GFP-LC3 positive cell was assessed by counting a minimum of 50 cells via Image ProPlus for duplicate samples per condition in three independent experiments. The significance of alterations in autophagy levels were determined by a two-tailed, heteroscedastic student's t-test, wherein p≦0.05 is considered significant.

Western Blot.

Expression levels of Flag-tagged Beclin 1, Bcl-X_{L} and M11 in MCF7 cells were verified by western blot analysis using commercial mouse monoclonal anti-Flag M2-peroxidase antibody (Sigma). As a loading control, the levels of Actin in MCF7 cell lysates were detected with mouse anti-actin (Chemicon).

### Results

Beclin 1 BH3D Binds in a Similar Manner to Both M11 and Bcl-X_{L}.

Beclin 1 is a key autophagy effector that is 99% conserved between humans and mice, with the human and mouse Beclin 1 BH3Ds sharing 100% amino acid identity. The Beclin 1 BH3D is the primary determinant of Beclin 1 binding to cellular and γHV Bcl-2s. The Beclin 1 BH3D binds to Bcl-2 homologs as an amphipathic α-helix, with the hydrophobic face of the helix buried in a hydrophobic groove on the surface of the Bcl-2 homolog. A comparison of co-complex structures of the Beclin 1 BH3D bound to M11 or Bcl-X_{L} demonstrates that each interaction involves the same Beclin 1 residues and buries 978 Å2 and 1052 Å2 of surface area at the interface, respectively, as calculated using PISA: In both structures BH3D residues L112, L116 and G120 are completely buried; F123 is partially buried; and G120 and D121 interact with a Gly-Arg pair conserved in most Bcl-2s, including Bcl-X_{L} and M11. G120 is packed against the conserved Bcl-2 Gly-Arg main chain, while the BH3D D121 makes a bidentate salt bridge with the conserved Bcl-2 Arg. Equivalents of these Beclin 1 residues are highly conserved amongst other BH3Ds.

However, despite their similar three-dimensional structures and mode of binding, M11 and Bcl-X_{L} share only 20.5% sequence identity and 53.8% sequence similarity. The differences in the residues lining the hydrophobic groove translate to differential affinities for various BH3D-containing proteins. Thus, the interactions of the Beclin 1 BH3D with M11 and Bcl-X_{L} provide a good model system for a detailed thermodynamic analysis to delineate differences in the determinants of binding to M11 and Bcl-X_{L}.

Qualitative co-immunoprecipitation analyses have shown that Beclin 1 binds to Bcl-2 and Bcl-X_{L}, but only weakly, or not at all to the other cBcl-2 homologs, Mcl-1, A1 and Bcl-W. The isolated Beclin 1 BH3D binds to different Bcl-2 proteins with diverse affinities in the micromolar range: a weak K_{d} of ˜54 mM to KSHV Bcl-2; ˜9 mM to Bcl-2; and a similar, moderate binding affinity of ˜1.5 mM to both γHV68 M11 and Bcl-X_{L} (Tables 1 and 4). Further, for both M11 and Bcl-X_{L} (Table 1), the favorable free energy of association (ΔG) is due to enthalpic contribution's (ΔHapp), rather than due to entropic contributions (ΔSapp), which is negative in each case. It was recently shown that the Beclin 1 BH3D is disordered in solution, and that BH3D residues 116-127 appear to serve as an “anchor” that nucleates concomitant folding and binding of the Beclin 1 BH3D to Bcl-2 homologs. Therefore, the negative ΔSapp likely reflects BH3D desolvation and increased structure upon binding, which proceeds despite the negative ΔSapp, due to enthalpic compensation. Despite the similar ΔG of binding to M11 and Bcl-X_{L}, entropic and enthalpic contributions to binding are different, with ΔHapp for binding to M11 being ˜2-fold higher, and TΔSapp being ˜4-fold lower than that for binding to Bcl-X_{L} (Table 1).

Specific Beclin 1 Mutations Abrogate Autophagy Down-Regulation by Bcl-X_{L} but not M11.

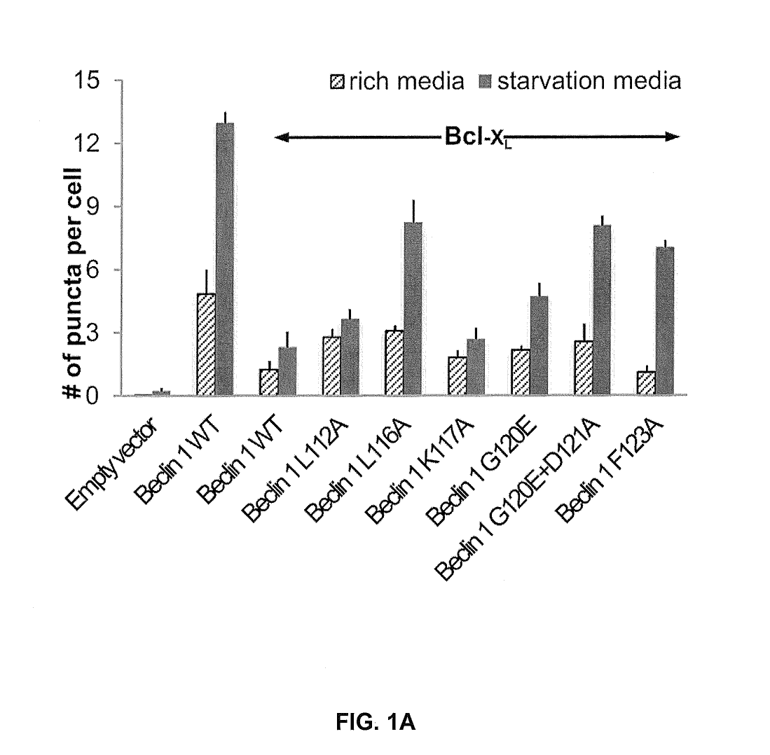

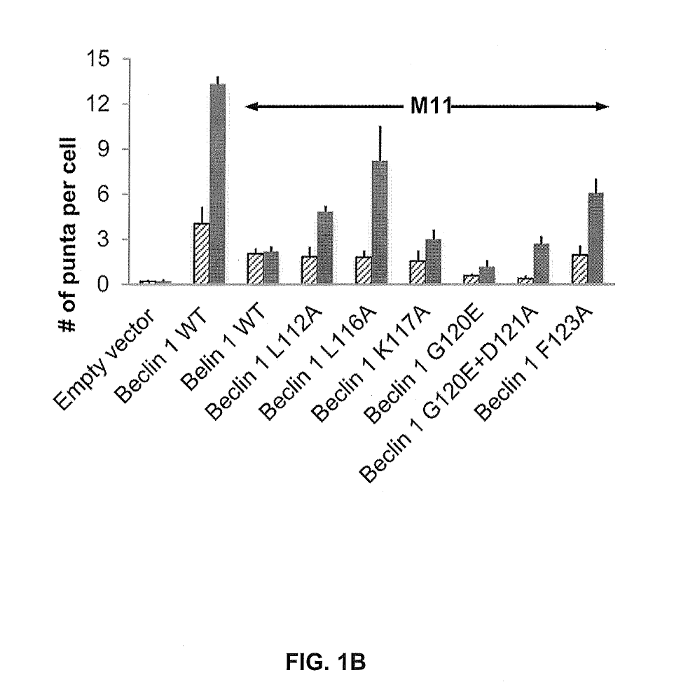

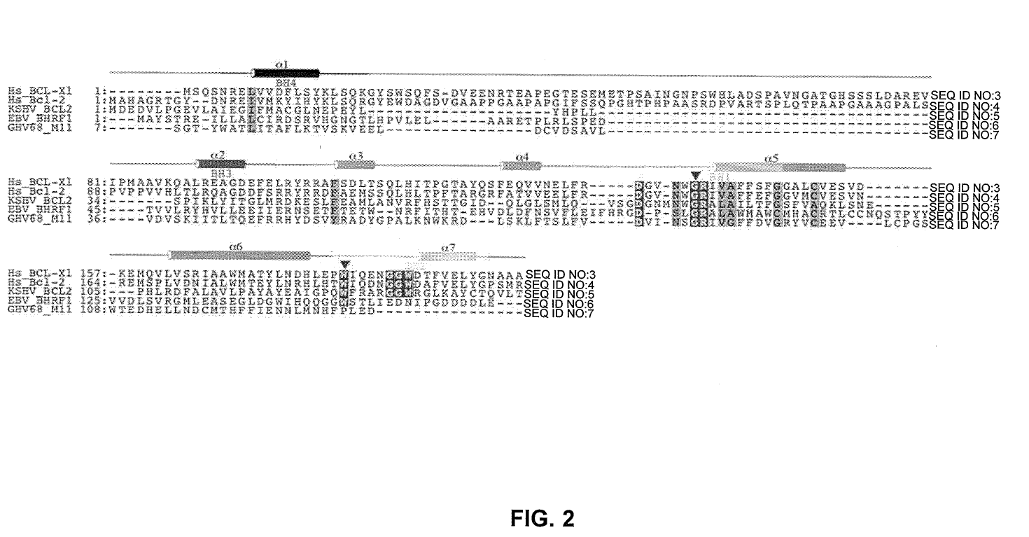

Based on the structures of the Beclin 1 BH3D bound to either M11 or Bcl-X_{L}, the down-regulation of autophagy mediated by Bcl-X_{L} and M11 was first assessed upon expression of the Beclin 1 BH3D single mutants: L112A, L116A, K117A, G120E and F123A. Autophagy levels were monitored by assaying the change in cellular localization of a transiently-expressed, GFP-tagged, mammalian autophagy-specific marker, LC3 (GFP-LC3), from a diffuse cytoplasmic distribution to localized punctae corresponding to autophagosomal structures (FIG. 1). These assays were performed using human MCF7 breast carcinoma cells, which express very low levels of Beclin 1 and do not show starvation-induced increases in autophagy unless Beclin 1 is ectopically expressed (FIG. 1). This allows the effect of Beclin 1 mutants in the absence of endogenous Beclin 1 to be monitored. The transient co-expression of either Bcl-X_{L} or M11 was then used to assay the ability of these homologs to inhibit autophagy mediated by the different Beclin 1 BH3D mutants (FIG. 1).

Transient expression of Beclin 1 in MCF7 cells leads to a marked increase in autophagy upon starvation (p=0.00060 for starved versus unstarved cells); and this starvation-induced, Beclin 1-dependent autophagy is significantly down-regulated by expression of either Bcl-X_{L} (p=0.00019 for Bcl-X_{L} versus empty vector; FIG. 1A) or M11 (p=0.00003 for M11 versus empty vector; FIG. 1B). M11 was found to inhibit starvation-induced autophagy at least as potently as Bcl-X_{L} (FIG. 1). Further, it was found that, in general, Beclin 1 BH3D mutations are less deleterious for the M11-mediated down-regulation of Beclin 1-dependent autophagy.

Under starvation conditions, Bcl-X_{L} was found to down-regulate autophagy mediated by the K117A Beclin 1 mutant as effectively as that mediated by WT Beclin 1 (p=0.5043 for mutant versus WT Beclin 1). However, Bcl-X_{L}-mediated downs regulation of autophagy is less pronounced upon expression of L112A (p=0.06209 for mutant versus WT Beclin 1) or G120E (p=0.01190 for mutant versus WT Beclin 1) Beclin 1 mutants (FIG. 1A). Amongst the Beclin 1 single mutants, the most significant abrogation of Bcl-X_{L}-mediated autophagy down-regulation was observed upon expression of the mutants F123A (p=0.00246 for mutant versus WT Beclin 1) and the L116A (p=0.00212 for mutant versus WT Beclin 1).

Similar to Bcl-X_{L}, expression of the Beclin 1 K117A mutant (p=0.15725 for mutant versus WT Beclin 1) does not affect M11-mediated down-regulation of autophagy (FIG. 1B). M11-mediated down-regulation of autophagy is significantly weaker upon expression of the mutants F123A (p=0.01070 for mutant versus WT Beclin 1) and L112A (p=0.00065 for mutant versus WT Beclin 1). Further, the most significant abrogation of M11-mediated autophagy down-regulation is observed when L116A mutant Beclin 1 was expressed (p=0.00432 for mutant versus WT Beclin 1). However, contrary to expectations from structural analysis, M11 effectively down-regulates autophagy upon expression of the G120E single mutant (p=0.03131 for mutant versus WT Beclin 1).

Interestingly, cellular co-immunoprecipitation assays had been used previously to show that a full-length Beclin 1 G120A+D120A mutant still binds to M11, suggesting that the M11 binding site could accommodate a small side chain at the G120 position, and the D120 side chain was dispensable for binding. Despite this, it was expected that the mutation of the BH3D G120 to the large and negatively-charged Glu residue would disrupt binding to both Bcl-X_{L} and M11, consequently abrogating the down-regulation of autophagy by these Bcl-2 homologs. However, the data (FIG. 1B) indicates that unlike BCl-X_{L}, the M11 binding site is flexible enough to accommodate the Glu side chain, and enables M11 to effectively down-regulate autophagy mediated by G120E Beclin 1.

It was then decided to examine the role of D121 in the context of the G120E mutation, by assaying the ability of Bcl-X_{L} and M11 to down-regulate autophagy mediated by a G120E+D121A Beclin 1 double mutant (DM). As expected, expression of G120E+D121A DM Beclin 1 resulted in abrogation of Bcl-X_{L}-mediated autophagy down-regulation (p=0.00079 for mutant versus WT Beclin 1), comparable to the effect seen upon expression of the L116A mutant Beclin 1 (FIG. 1A). Strikingly however, and in complete contrast to Bcl-X_{L}, it was found that M11 effectively down-regulates autophagy mediated by the G120E+D121A Beclin 1 DM (p=0.22842 for mutant versus WT Beclin 1) (FIG. 1B). Thus, a G120E+D121A double mutation prevents abrogation of autophagy by Bcl-X_{L}, but not by M11.

Identification of peptides that bind to M11, but not to Bcl-X_{L}.

Differential specificity determinants that are either required for binding, or prevent binding, to either M11 or Bcl-X_{L} were established by using ITC to quantify and compare binding of a systematic set of Beclin 1 BH3D-derived peptides to Bcl-X_{L} and M11. These peptides have alterations in residues known to be involved in binding: three hydrophobic residues, L112, L116, and F123 and a basic residue, K117; which were changed to Ala while G120 was changed to Glu (Table 1). Each of these residues is conserved amongst BH3D domains and buried in the interaction interface with Bcl-2s.

In general, each residue change impacted binding to Bcl-X_{L} more than to M11; with the different residue changes having very diverse thermodynamic effects on binding to either M11 and Bcl-X_{L} (Table 1). All the changes weakened binding to Bcl-X_{L}, but not to M11. The L112A mutation weakened binding to Bcl-X_{L} to barely detectable levels, but reduced binding to M11 by only ˜3-fold; while the F123A mutation weakened binding to Bcl-X_{L} by ˜10-fold, and to M11 by ˜4-fold. Interestingly, although the K117A mutant appears to weaken binding to Bcl-X_{L}˜10-fold, it actually improves binding to M11 by ˜2-fold. Lastly, none of the residue changes abolished binding to M11, but two single residue changes, L116A and G120E, that completely abrogate binding to Bcl-X_{L}, were identified (Table 1).

These two changes were also the most deleterious for binding to M11, and strikingly, are part of the Anchor region that was recently identified within the BH3D. The L116A and G120E mutations reduce binding affinity for M11 more than 70-fold and 26-fold respectively (Table 1).

Contrary to initial expectations based on the structure of the WT BH3D bound to M11; but consistent with the cellular autophagy assays, the G120E mutant is still able to bind to M11 (Table 1), although with 26-fold weaker affinity. Therefore, consistent with cellular experiments, the M11 binding site is sufficiently flexible to allow binding of the E120 residue, and may stabilize E120 by electrostatic interactions with the conserved M11 R87, which also interacts with the peptide D121. Further, it was hypothesized that this likely causes competition for the R87 interaction between the carboxylates of these two residues, resulting in the reduced binding affinity of the G120E peptide for M11; and that perhaps binding would be enhanced by changing the D120 to an Ala (i.e. a G120E+D121A DM peptide). Consistent with this hypothesis and the cellular experiments in the previous section, it was discovered that, while a G120E+D121A DM peptide does not bind to Bcl-X_{L}, it binds to M11 with only ˜4.7-fold weaker affinity compared to the WT and ˜5.7-fold better compared to the G120E peptide. It is likely that binding of the G120E peptide to M11 involves some competition between the G120E and D121 to side chains for the M11 R87 electrostatic interaction; however, this competition is eliminated in the G120E+D121A DM peptide, enabling it to bind better than the G120E peptide.

Structure of the DM Peptide Bound to M11.

In order to elucidate the mechanism by which M11 can still bind to the DM peptide, the X-ray crystal structure of the M11-DM peptide complex was determined. Residues altered in the DM peptide, E120 and A121, have very well defined electron density. It was discovered that the WT BH3D and DM peptide both bind by a similar mode in the M11 hydrophobic surface groove. The two complexes superimpose with an RMSD of 0.451 Å over 148 Ca atoms, indicating that they are fairly similar; although the superposition is somewhat worse than that of the two complexes within the asymmetric units of structures of either the M11-DM peptide complex (0.162 Å) or the WT BH3D complex (0.031 Å). Despite this similarity of interaction, the total surface area buried in the interaction interface is significantly reduced in the M11-DM peptide complex, to 868 Å2; compared to 978 Å2 in the M11-WT BH3D complex. This reduced buried surface area likely accounts for the reduced binding affinity and is the result of the more substantial side chain conformational changes in the bound DM peptide relative to the WT BH3D as well as subtle compensatory changes in M11 that facilitate binding to the DM peptide.

Separate superimpositions of the M11 molecule in each complex indicates that there is not much conformational change in the M11 structure, with RMSDs of 0.38 Å over 130 Ca atoms; although the superposition is somewhat worse than that of the two M11 subunits within the asymmetric units of structures of either the M11-DM peptide complex (0.17 Å) or the WT BH3D complex (0.03 Å). Interestingly maximal conformational change is seen not at the BH3D binding groove, but rather at the α1-α2 loop, which is structurally analogous to the phosphorylation loop of Bcl-X_{L} and Bcl-2.

Significant changes are seen in the bound DM peptide, compared to the WT BH3D. The bound WT and DM BH3Ds in the two structures superimpose with an RMSD of 0.99 Å over 18 Ca atoms, with the comparatively poorer alignment chiefly attributable to the slightly shifted positions of residues 117-125. The identical N-terminal halves of the two peptides superimpose fairly well between the WT BH3D and DM peptide structures, with an RMSD of 0.38 Å2 over 9 Ca atoms. However, superimposition of the C-terminal half is poorer, with an RMSD of 1.35 Å2 over 9 Ca atoms. Thus, binding to M11 is enabled by significant shifts of the DM peptide main chain, especially of its C-terminal half, (FIG. 3B), relative to WT BH3D.

Differences in the Interactions of the WT BH3D and DM Peptide with M11.

Peptide amino acids corresponding to BH3D residues L112 and L116 bind in similar locations in the WT BH3D and DM peptide complexes, with pairwise differences in Ca positions being 0.4 Å and 0.5 Å respectively. The packing of L112 is virtually identical in each complex, with L112 being sandwiched between M109 and L116, which are approximately one helical turn away on each side within the peptide, and surrounded by a hydrophobic pocket lined by M11 residues Y60, A63 and L74. Similarly, in each complex, L116 is packed into a hydrophobic pocket lined by M11 residues F48, Y60, L78, and V94, although there are some subtle differences in the atomic details of the interaction.

Starting at K117, there are incrementally increasing shifts in DM peptide residue positions relative to those in WT BH3D. The pair wise shift at K117 Cα is 1.1 Å, which enables additional interactions between K117 and M11 in the DM complex. The aliphatic part of K117 packs against aliphatic chains of M11 D81 and R87 in both complexes, but in the DM complex also with M11 L78 and S77. Further, while the K117 amino group does not make any interactions in the WT complex, in the DM complex it electrostatically bonds with the M11 D81 carboxylate and hydrogen bonds the S77 hydroxyl. Similarly, the next peptide residue, V118, is solvent exposed and does not interact with M11 in the WT BH3D complex, but a 1.3 Å Ca shift at this position in the DM peptide complex relative to the WT BH3D, results in packing against M11 Y56. The following residue, T119, has a smaller Ca shift between the WT BH3D and DM peptide, and maintains similar, but slightly different, interactions in both complexes, with the aliphatic parts of the side chain packed against M11 residues F48, Y52 and the H51 main chain.

The next two residues are altered in the DM peptide: E120 and A121, compared to G120 and D121 in the WT BH3D. The incremental shifts preceding these residues results in maximal shifts of 1.1 Å at the E120 Cα from the WT BH3D G120 Cα position, and of 2.0 Å at the A121 Cα compared to WT BH3D D121 Cα position. At the A121 Cα, this shift corresponds to approximately half a helical turn relative to the WT BH3D-M11 complex. In the M11-WT BH3D complex, the G120-D121 main chain packs in an anti-parallel manner against the main chain of two conserved M11 residues: G86 and R87. In contrast, in the M11-DM peptide complex, E120 extends across the M11 hydrophobic groove, with the aliphatic part of the side chain packed against the M11 G86 main chain, the aliphatic parts of R87 and F48, to make one salt bridge with M11 R87. Similarly, while in the WT BH3D complex, the D121 side chain is stabilized by packing against the aliphatic part of R87, and a bidentate salt bridge to M11 R87; A121 in the DM peptide complex makes no contacts with M11, and is completely solvent exposed, as a consequence of these main chain shifts. Thus, the main chain shifts of the DM peptide enable the E120-M11 R87 interaction and remove A121 from M11 interactions.

L122, the peptide residue following two altered residues, is also significantly shifted and has completely different environments in the WT BH3D and DM peptide structures. In the WT, L122 is solvent exposed and makes no contacts with M11; while in the DM peptide complex it is packed against M11 H51 and V55. Pairwise Ca shifts between the WT and DM peptide structures decrease to 0.7 Å at F123, which allows the side chain to bind in equivalent M11 hydrophobic surface pockets comprised of residues L44, E47, F48, H51, G86 and V89 in each complex, but with an altered orientation of the F123 aromatic ring and subtly different interactions. The relative shifts between WT BH3D and DM peptide are retained at the D124 Cα position. This allows the aliphatic part of the D124 side chain to pack against the G86 Cα in both complexes; as well as with the aliphatic parts either of M11 N84 in the WT BH3D, or of E120 in the DM peptide complex. Further, in the WT BH3D, the D124 carboxylate group hydrogen bonds to the G86 amide, but not in the DM peptide.

A TAT-DM Peptide Selectively Abrogates Down-Regulation of Autophagy by M11, but not Bcl-X_{L}.

Lastly, whether the DM peptide would specifically prevent M11-mediated down-regulation, but not Bcl-X_{L}-mediated down-regulation of Beclin 1-dependent autophagy in cells was investigated. To make the peptide cell-permeable, the HIV-1 trans-activating transcriptional activator protein transduction domain (TAT) was attached via a diglycine linker to the N-terminus of the DM peptide.

Treatment of MCF7 cells with TAT-DM peptide did not increase levels of autophagy in either nutrient-rich or starvation conditions. As noted in the first section, ectopic Beclin 1 expression mediates autophagy, and autophagy levels are further elevated by starvation, while transient expression of either Bcl-X_{L} or M11 results in down-regulation of autophagy in both nutrient-rich and starvation conditions. TAT-DM peptide treatment of cells that express Beclin 1, but not Bcl-X_{L} or M11, causes only a slight elevation in autophagy levels relative to untreated cells (p=0.03383 for treated versus untreated cells). TAT-DM peptide treatment of cells that transiently express Bcl-X_{L} in addition to Beclin 1 had an insignificant effect compared to untreated cells (p=0.92294 for treated versus untreated cells), presumably because the TAT-DM peptide does not bind to Bcl-X_{L}, preventing Bcl-X_{L} from down-regulating autophagy. Strikingly however, DM peptide treatment of cells that transiently express M11 in addition to Beclin 1, markedly increases autophagy levels compared to untreated cells (p=0.0010 for treated versus untreated cells), indicating that the TAT-DM peptide specifically bound to M11, preventing M11 from binding to Beclin 1 and from down-regulating Beclin 1-mediated autophagy. Thus, the TAT-DM peptide inhibits M11-mediated autophagy down-regulation of autophagy, but not Bcl-X_{L}-mediated down-regulation of autophagy.

### DISCUSSION

In this study, a mutational analysis of Beclin 1 was used to identify differential selectivity determinants that prevent Bcl-X_{L}, but not M11, from binding to Beclin 1 and down-regulating autophagy. Based on this information, a peptide that binds specifically with moderate affinity to M11, but not to Bcl-X_{L} was designed. The co-complex structure of this peptide bound to M11 was determined to elucidate the mechanism by which M11, but not Bcl-X_{L}, binds to this peptide. Subtle M11 conformational changes facilitated by the flexibility of the M11 binding groove, combined with substantial main chain shifts and side chain conformational changes in the DM peptide relative to the WT BH3D, was found to allow the DM peptide to bind to M11. All of this information is essential to understanding the atomic bases of differential specificity of M11 and cBcl-2s. Lastly, a cell-permeable version of this peptide was designed that prevents the M11-mediated down-regulation of autophagy, but does not prevent the Bcl-X_{L}-mediated down-regulation of autophagy. This peptide can be used to study the role of M11 at different stages of the γHV68 life-cycle, by treating virus infected cells with the peptide at different time-points and assaying the effects on the viral infection.

The effect of mutations on binding, combined with the structural information not only helps explain the atomic bases of the differential specificity, but also provides direct information on how M11-BH3D interactions may be targeted in vivo. Identifying determinants specific for binding of BH3Ds to γHV Bcl-2s, provides information about their ability to bind to different BH3D-containing proteins, and consequently their ability to differentially regulate pathways impacted by these BH3D-containing proteins. Finally, all of this information will be invaluable for the rational design of small molecules that can selectively inhibit M11 and other γHV Bcl-2s, but not cBcl-2s. Such a small molecule would be an invaluable tool to study the interactions and roles of v-Bcl-2s not only in cell culture, but also in vivo. A more specific inhibitor that can selectively inhibit a given Beclin 1 γHV Bcl-2 interaction, removing only the γHV blockade of autophagy would be an extremely useful tool to study the effect of xenophagy in regulating γHV infections. This information will help in the investigation of the role of γHV Bcl-2s in the infectious cycle of these viruses. Ultimately, such small molecule inhibitors may even form the basis of novel therapeutics to treat γHV infection by promoting the autophagic degradation of viruses, apoptotic destruction of infected host cells or restoration of the tumor suppressor activity of Beclin 1. Thus, this research will substantially inform future research on the pathogenesis of infections caused by γHVs.

While the disclosure is susceptible to various modifications and alternative forms, specific exemplary embodiments of the present invention have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

TABLE 1
Thermodynamic parameters for binding of different Beclin 1 BH3D mutants to M11 and Bcl-X_{L}.

| | M11 | | | | Bcl-X_{L} | | | |
|---|---|---|---|---|---|---|---|---|
| Beclin 1 | K_{d} | ΔH | ΔG | ΔS | K_{d} | ΔH | ΔG | ΔS |
| BH3D | (μM) | (kJ/mol) | (kJ/mol) | (J/K · mol) | (μM) | (kJ/mol) | (kJ/mol) | (J/K · mol) |
| WT | 1.38 ± 0.41 | −70.97 ± 6.39 | −33.51 ± 0.71 | −125.72 ± 22.53 | 1.95 ± 0.19 | −43.57 ± 1.44 | −32.58 ± 0.23 | −36.89 ± 4.53 |
| L112A | 4.33 ± 0.86 | −47.36 ± 1.53 | −30.63 ± 0.53 | −56.14 ± 4.10 | 109.71 ± 3.10 | −42.36 ± 1.07 | −22.59 ± 0.07 | −66.34 ± 3.84 |
| L116A | 177.62 ± 16.95 | −36.40 ± 4.64 | −21.40 ± 0.24 | −50.33 ± 16.36 | No | — | — | — |
| | | | | | Binding | | | |
| K117A | 0.66 ± 0.17 | −69.08 ± 3.05 | −35.32 ± 0.67 | −113.29 ± 11.74 | 18.93 ± 4.15 | −37.66 ± 3.72 | −26.99 ± 0.58 | −35.83 ± 11.52 |
| G120E | 36.49 ± 8.10 | −52.17 ± 5.81 | −25.36 ± 0.52 | −89.97 ± 20.91 | No | — | — | — |
| | | | | | Binding | | | |
| F123A | 5.20 ± 1.53 | −59.36 ± 2.72 | −30.20 ± 0.74 | −97.84 ± 11.61 | 407.56 ± 75.68 | −30.70 ± 8.28 | −19.36 ± 0.46 | −38.04 ± 29.34 |
| G120E + | 6.43 ± 0.15 | −62.34 ± 2.72 | −29.62 ± 0.06 | −109.81 ± 9.28 | No | — | — | — |
| D121A | | | | | Binding | | | |

TABLE 2
Summary of crystallographic data statistics.

| | |
|---|---|
| Wavelength (Å) | 0.97934 |
| Data range (Å) | 1.8-50.00 |
| Mosaicity | 0.329-0.633 |
| Unique | 19337 |
| reflections | |
| Avg. multiplicity | 3.8 (3.2) |
| Completeness | 99.3 (91.7) |
| (%) | |
| Rsym (%) | 5.0 (14.9) |
| I/σₗ | 9.1 |

Values in parentheses pertain to the outermost shell of data.
^{‡}R_{sym} = Σ_{h,l}|l_{h,l} − <lₕ>|/Σ_{h,l} l_{h,l}.

TABLE 3
Summary of crystallographic refinement statistics.

| Model: | |
|---|---|
| M11 Monomer A residues | 135 |
| M11 Monomer B residues | 136 |
| Beclin 1 Monomer C residues | 20 |
| Beclin 1 Monomer D residues | 22 |
| Water molecules | 133 |
| Sulfate molecules | 4 |
| Data Range (Å) | 50-2.1 |
| R_{work} (%) | 16.0 |
| R_{free} (%) | 22.4 |
| Average B-values (Å²) | 34.7 |
| Main Chain | 26.7 |
| Side Chain | 28.7 |
| Water | 48.0 |
| All Atoms | 34.7 |
| B-factor RMSDs between bonded | |
| atoms: | |
| Main chain | 2.332 |
| Side Chain | 4.026 |
| RMSDs from target values: | |
| Bond Lengths | 0.020 |
| Bond Angles | 1.985 |
| Dihedral Angles | 21.33 |
| Improper Angles | 1.91 |
| Cross-validated sigma coordinate | 0.24 |
| error (Å) | |
| Ramachandran outliers | 0 |

^{‡}R factor = Σ_{h,}|F_{obs} − |F_{calc}|/Σₕ|F_{obs}|. Test set for R_{free} consisted of 5.5% of data.

TABLE 4
Differential binding of Beclin 1 BH3 domain related inhibitory
peptides to various Bcl-2 proteins.

| Peptides | Bcl-2 | Bcl-X_{L} | γHV Bcl-2 |
|---|---|---|---|
| WT | 10.4 ± 3.5 | 1.9 ± 0.2 | 1.4 ± 0.4 |
| L112A | No Binding | 109.7 ± 3.1 | 4.3 ± 0.9 |
| L116A | No Binding | No Binding | 177.6 ± 16.9 |
| K117A | 24.5 ± 6.0 | 18.9 ± 4.2 | 0.7 ± 0.2 |
| G120E | No Binding | No Binding | 36.5 ± 8.1 |
| D121A | Not done | No Binding | 1.3 ± 0.7 |
| G120E + D121A | No Binding | No Binding | 6.4 ± 0.2 |
| (DS peptide) | | | |
| F123A | No Binding | 407.6 ± 75.7 | 5.2 ± 1.5 |

What is claimed is:

1. A synthetic peptide derivative of a BH3 domain comprising one or more substitutions at amino acid positions conserved among BH3 domains, the synthetic peptide having a higher affinity for a viral Bcl-2 than its affinity for a cellular Bcl-2.

2. The synthetic peptide derivative of claim 1, wherein the affinity of the synthetic peptide for the viral Bcl-2 is at least 5 times higher than its affinity for the cellular Bcl-2.

3. The synthetic peptide derivative of any one of claim 1 or 2, wherein the synthetic peptide does not detectably bind to cellular Bcl-2.

4. The synthetic peptide derivative of any one of claims 1-3, wherein the synthetic peptide alters the effect of viral Bcl-2 on a cellular pathway.

5. The synthetic peptide derivative of any one of claims 1-4, wherein the synthetic peptide alters the effect of viral Bcl-2-mediated suppression of autophagy or apoptosis.

6. The synthetic peptide derivative of any one of claims 1-5, wherein, the synthetic peptide inhibits down-regulation of autophagy by a viral Bcl-2 homolog.

7. The synthetic peptide derivative of any one of claims 1-6, wherein, the synthetic peptide comprises a substitution at one or more amino acid positions conserved among BH3 domains.

8. The synthetic peptide derivative of claim 7, wherein the one or more amino acid substitutions includes a substitution that changes a hydrophobic amino acid or a basic amino acid to an alanine residue and/or changes a glycine residue to a polar or acidic amino acid.

9. The synthetic peptide derivative of claim 7, wherein the synthetic peptide comprises one or more amino acid substitutions selected from at amino acid positions L112, L116, K117, G120, D121, and F123 relative to wild type Beclin 1 BH3 domain.

10. The synthetic peptide derivative of claim 9, wherein the synthetic peptide comprises one or more amino acid substitutions selected from L112A, L116A, K117A, G120E, D121A, and F123A.

11. The synthetic peptide derivative of claim 7, wherein the synthetic peptide is a synthetic peptide homolog of Beclin 1 BH3 domain and comprises one or more substitutions corresponding to position L8, L12, K13, G16, D17, or F19 of SEQ ID NO:1.

12. The synthetic peptide derivative of claim 11, the synthetic peptide derivative comprising substitutions G16E and D17A relative to SEQ ID NO:1.

13. The synthetic peptide derivative of claim 12 comprising SEQ ID NO:2 or its reverse sequence.

14. The synthetic peptide derivative of any one of claims 1-13, wherein the synthetic peptide comprises at least one D amino acid.

15. The synthetic peptide derivative of any one of claims 1-14, wherein one or more peptide bonds of the synthetic peptide is substituted with a non-peptide bond.

16. The synthetic peptide derivative of any one of claims 1-15, further comprising a cell-penetrating peptide sequence.

17. The synthetic peptide derivative of any one of claims 1-16, further comprising a cell-targeting moiety.

18. A therapeutic composition comprising the synthetic peptide derivative of any one of claims 1-17 and a pharmaceutically acceptable excipient.

19. A method of treating a person infected with a γHV in need of treatment, the method comprising administering to the person an amount of the synthetic peptide derivative of any one of claims 1-17 or the therapeutic composition of claim 18 effective to ameliorate one or more symptoms of the γHV infection.
